SEQUENCE LISTING (16 sequences)

SEQ ID NO: 1 - protein, 12 aa, synthetic construct
KKLFKKILKYLK

SEQ ID NO: 2 - protein, 12 aa, synthetic construct
RRLFRRILRYLR

SEQ ID NO: 3 - protein, 12 aa, synthetic construct
RFLFRLILRYLR

SEQ ID NO: 4 - protein, 12 aa, synthetic construct
RRLFRLILRYLF

SEQ ID NO: 5 - protein, 12 aa, synthetic construct
RKLFKRILRYLK

SEQ ID NO: 6 - protein, 12 aa, synthetic construct
KRLFRKILKYLR

SEQ ID NO: 7 - protein, 12 aa, synthetic construct
KRLFKRILRYLK

SEQ ID NO: 8 - protein, 12 aa, synthetic construct
RKLFRKILKYLR

SEQ ID NO: 9 - DNA, 36 bp, synthetic construct
  source 1..36: mol_type = other DNA
aaaaaactgtttaaaaaaattctgaaatatctgaaa

SEQ ID NO: 10 - DNA, 36 bp, synthetic construct
  source 1..36: mol_type = other DNA
cgccgcctgtttcgccgcattctgcgctatctgcgc

SEQ ID NO: 11 - DNA, 36 bp, synthetic construct
  source 1..36: mol_type = other DNA
cgctttctgtttcgcctgattctgcgctatctgcgc

SEQ ID NO: 12 - DNA, 36 bp, synthetic construct
  source 1..36: mol_type = other DNA
cgccgcctgtttcgcctgattctgcgctatctgttt

SEQ ID NO: 13 - DNA, 36 bp, synthetic construct
  source 1..36: mol_type = other DNA
cgcaaactgtttaaacgcattctgcgctatctgaaa

SEQ ID NO: 14 - DNA, 36 bp, synthetic construct
  source 1..36: mol_type = other DNA
aaacgcctgtttcgcaaaattctgaaatatctgcgc

SEQ ID NO: 15 - DNA, 36 bp, synthetic construct
  source 1..36: mol_type = other DNA
aaacgcctgtttaaacgcattctgcgctatctgaaa

SEQ ID NO: 16 - DNA, 36 bp, synthetic construct
  source 1..36: mol_type = other DNA
cgcaaactgtttcgcaaaattctgaaatatctgcgc

(19) United States
(12) Patent Application Publication
(10) Pub. No.: US 2025/0011364 A1
(43) Pub. Date: Jan. 9, 2025

(54) TUMOR TISSUE-PENETRATING PEPTIDE, SPECIFICALLY BINDING TO NEUROPILIN 1, FOR DRUG DELIVERY TO TUMOR, AND USE THEREOF

(71) Applicant: CELLICON LAB INC., Daejeon (KR)

(72) Inventors: Min Gyu SOUNG, Daejeon (KR); Bo Kyung PARK, Daejeon (KR); Won Suk CHOI, Daejeon (KR); Young Pil JEONG, Daejeon (KR); Mi Yeong AN, Daejeon (KR); Jae Hoo PARK, Daejeon (KR)

(73) Assignee: CELLICON LAB INC., Daejeon (KR)

(21) Appl. No.: 18/712,400

(22) PCT Filed: Jul. 15, 2022
(86) PCT No.: PCT/KR2022/010396

§ 371 Date: May 22, 2024

(30) Foreign Application Priority Data
Mar. 31, 2022 (KR) 10-2022-0040531

(51) Int. Cl. C07K 7/08; A61K 38/00; A61K 47/42; A61K 47/64

(52) U.S. Cl. CPC C07K 7/08; A61K 47/42; A61K 47/64; A61K 38/00

(57) ABSTRACT

The tumor tissue-penetrating peptide of the present invention, a derivative thereof, or a fragment thereof effectively delivers a drug deep into tumor or tissues through an assembling or fusion method and exhibits not only excellent penetration activity into biomembranes but also excellent tumor selectivity to maximize anticancer activity, and thus the tumor tissue-penetrating peptide, a derivative thereof, or a fragment thereof can be widely utilized as an active ingredient in pharmaceutical compositions for treatment or prevention of and compositions for diagnosis of cancer or angiogenesis-related diseases, which exhibit improved tumor selectivity and delivery ability to tumor and target cancer tissues or cells including biomembranes.

### TECHNICAL FIELD

The present invention relates to a tumor tissue-penetrating peptide that binds to neuropilin 1, a derivative thereof, or a fragment thereof; a polynucleotide encoding the tumor tissue-penetrating peptide, a derivative thereof, or a fragment thereof; a pharmaceutical composition for treatment or prevention of and a composition for diagnosis of cancer or angiogenesis-related diseases, containing a tumor tissue-penetrating peptide that binds to neuropilin 1, a derivative thereof, or a fragment thereof; and uses thereof.

### BACKGROUND ART

Cancer is one of the most common diseases worldwide, and currently commonly performed treatments include chemotherapy, surgery, radiation therapy, hematopoietic stem cell transplantation, and immunotherapy, and studies on the molecular mechanisms of cancer are actively underway, but a large number of the currently developed treatments rely on surgery.

Until now, therapeutic agents using antigens specific to tumor cells and antibodies targeting these have mainly been developed. However, antibodies cause serious side effects because of problems such as concerns about immune response and low efficiency of penetration into tissues. In particular, therapeutic agents for solid cancers have a relatively low response rate compared to that of therapeutic agents for blood cancer, this makes it more difficult to treat solid cancers, and one of the reasons for this is the physiological properties of tumor tissues. Physiological factors that interfere with the penetration and distribution of antibodies into tumor tissues may be classified into four: endothelial barrier, high interstitial fluid pressure in tumor tissues, stromal impediment, and epithelial barrier.

Among these, the stromal impediment is an extracellular matrix barrier encountered when antibodies escape into microvessels and are convected to tissues. It is mainly composed of collagen and hyaluronan, and depending on the composition thereof, there is a site where the drug is well distributed and a site where the drug is not well distributed, causing uneven drug distribution. When the expression level of the extracellular matrix increases, cell density increases by solid stress, making drug delivery more difficult.

In order to overcome these problems, there is a case in which the drug delivery effect is improved by reducing the cell density in tumor tissues by inducing the death of tumor tissue cells and by reducing the solid stress by treating tumor tissues with collagenase, which decomposes collagen in tumor tissues (Eikenes et al. 2004).

As described above, numerous efforts have been made worldwide to develop anticancer drugs, but anticancer drugs that are free from problems such as selective delivery to cancer cells, in vivo stability, drug resistance, and side effects have not yet been developed. Therefore, there is an urgent need to develop anticancer drugs that can secure long-term stability in vivo and minimize side effects by targeting and selectively eliminating cancer cells.

### DISCLOSURE

### Technical Problem

In order to solve the problems of existing anticancer drugs, such as a low drug delivery rate, low penetrability into tumor membranes, low cancer cell selectivity, and various side effects, the present inventors have used a peptide exhibiting high affinity for neuropilin 1, which is overexpressed in tumor vascular cells and tumor cells. They found out that the peptide is more selective for cancer cells and exhibits superior penetrability into tumor tissues than existing anticancer drugs, and thus has a significantly excellent drug delivery rate, and have completed the present invention.

### Technical Solution

An object of the present invention is to provide a composition for drug delivery, comprising a tumor tissue-penetrating peptide, a derivative thereof, or a fragment thereof.

Another object of the present invention is to provide a carrier for drug delivery, comprising the tumor tissue-penetrating peptide, a derivative thereof, or a fragment thereof.

Still another object of the present invention is to provide a composition for delivery of a physiologically active substance to tumor, comprising the tumor tissue-penetrating peptide, a derivative thereof, or a fragment thereof.

Still another object of the present invention is to provide a pharmaceutical composition comprising the composition for drug delivery.

Still another object of the present invention is to provide a quasi-drug composition comprising the composition for drug delivery.

Still another object of the present invention is to provide a food composition comprising the composition for drug delivery.

Still another object of the present invention is to provide a feed composition comprising the composition for drug delivery.

Still another object of the present invention is to provide a composition for diagnosis of cancer, comprising a tumor tissue-penetrating peptide, a derivative thereof, or a fragment thereof.

Still another object of the present invention is to provide a composition for diagnosis of angiogenesis-related disease, comprising a tumor tissue-penetrating peptide, a derivative thereof, or a fragment thereof.

### Advantageous Effects

The tumor tissue-penetrating peptide of the present invention, a derivative thereof, or a fragment thereof effectively delivers a drug deep into tumor or tissues through an assembling or fusion method and exhibits not only excellent tumor selectivity but also excellent penetration activity into tumor and tissues to maximize physiological activity, and thus the tumor tissue-penetrating peptide, a derivative thereof, or a fragment thereof can be widely utilized as an active ingredient in pharmaceutical compositions targeting tumor and tissues.

### BRIEF DESCRIPTION OF DRAWINGS

FIG. 1 is a diagram illustrating the results of examining the cancer cell killing effect by the MTT assay in order to analyze the effect of peptides of SEQ ID NO: 1 to SEQ ID NO: 8 on improving the anticancer efficacy of a physiologically active substance compared to a known peptide;

FIG. 2 is a diagram illustrating the results of analysis by the saturation ELISA binding assay in order to examine the binding ability of tumor tissue-penetrating peptides of the present invention to NRP-1;

FIG. 3 is a diagram illustrating the results of analyzing the effect of the tumor tissue-penetrating peptide of the present invention on improving the anticancer efficacy of a physiologically active substance through an in vivo anticancer experiment;

FIG. 4 is a diagram illustrating the results of analyzing the effect of the tumor tissue-penetrating peptide of the present invention on improving the tumor tissue-penetrating ability of a physiologically active substance;

FIG. 5 is a diagram illustrating the results of analyzing the effect of the tumor tissue-penetrating peptide of the present invention on improving the tumor tissue-penetrating ability of various types of physiologically active substances; and

FIG. 6 is a diagram illustrating the results of analyzing the effect of the tumor tissue-penetrating peptide of the present invention on inhibiting the binding ability of VEGF 165 to NRP-1.

### BEST MODE FOR CARRYING OUT THE INVENTION

Hereinafter, the contents of the present invention will be described in detail as follows. Meanwhile, the description and embodiment of an aspect disclosed in the present invention may also be applied to the description and embodiment of other aspects with respect to common matters. Additionally, all combinations of the various elements disclosed in the present invention fall within the scope of the present invention. In addition, the scope of the present invention cannot be considered limited to the specific description described below.

An aspect of the present invention provides a composition for drug delivery, containing a tumor tissue-penetrating peptide consisting of any one of amino acid sequences of SEQ ID NO: 5 to SEQ ID NO: 8, a derivative thereof, or a fragment thereof.

Another aspect of the present invention provides a composition for delivery of a physiologically active substance to tumor, containing a tumor tissue-penetrating peptide consisting of any one of amino acid sequences of SEQ ID NO: 5 to SEQ ID NO: 8, a derivative thereof, or a fragment thereof.

In the present invention, “tumor tissue-penetrating peptide” refers to a peptide that can deliver a physiologically active substance such as a drug to tumor. The tumor tissue-penetrating peptide may deliver a physiologically active substance to tumor cells and tissues.

The tumor tissue-penetrating peptide of the present invention may include a peptide consisting of any one of amino acid sequences of SEQ ID NO: 5 to SEQ ID NO: 8, a derivative thereof, or a fragment thereof.

The tumor tissue-penetrating peptide technology of the present invention is a cutting-edge biotechnology that greatly improves the absorption rate of raw materials in the body and increases bioavailability, is a next-generation new biomaterial platform technology that can fully demonstrate the efficacy when only an extremely small amount is introduced into cutting-edge pharmaceutical raw materials, and can be applied to the development of tumor tissue-penetrating drugs and quasi-drugs and the like.

The present invention may allow drugs that are difficult to be delivered into tumor due to low drug delivery rate, penetrability into tumor membranes, and cancer cell selectivity characteristics to permeate tumor and tissues and efficiently introduce the drugs, may be a pharmaceutical composition for treatment or prevention of and a composition for diagnosis of cancer or angiogenesis-related diseases, which exhibit improved tumor selectivity and tumor tissue-penetrating ability compared to existing tumor tissue-penetrating peptides, and uses thereof, and may be a tumor tissue-penetrating peptide that delivers drugs into tumor and tissues through an assembling or fusion method.

As an exemplary embodiment, the tumor tissue-penetrating peptide of the present invention may be prepared using a peptide exhibiting high affinity for neuropilin 1 expressed in tumor vascular cells and tumor cells, and exhibit greater cancer cell selectivity, penetrability into tumor membranes, and penetrability into tumor tissues than existing anticancer drugs.

As an exemplary embodiment, the tumor tissue-penetrating peptide of the present invention may exhibit tumor selectivity, but is not limited thereto.

As an exemplary embodiment, the tumor tissue-penetrating peptide of the present invention may have binding ability to neuropilin 1 (NRP-1), but is not limited thereto.

In the present invention, “neuropilin 1 (NRP-1)” is a transmembrane glycoprotein that binds to VEGF family ligands and semaphorin family ligands and is known to be very slightly expressed in normal cells but overexpressed in most tumor vascular endothelial cells, solid cancer cells, and hematologic tumor cells. (Bruder et al., 2004; Loser et al., 2005). Neuropilin 1 is known to act as a coreceptor of VEGFR1, VEGFR2, and VEGFR3, bind to various VEGF ligands, and thus be involved in angiogenesis, cell survival, migration & adhesion, and invasion in tumor tissues. Recently, it has been reported that neuropilin 1 can independently activate vascular permeability by VEGF165A, but the exact mechanism for this has not yet been revealed (Lise Roth et al. 2016).

In the present invention, “derivative” refers to a derivative of a tumor tissue-penetrating peptide consisting of any one of amino acid sequences of SEQ ID NO: 5 to SEQ ID NO: 8, and may be a peptide having an amino acid sequence in which some functional groups are added or some amino acid sequences are deleted, modified, substituted, or added to the amino acid sequences of SEQ ID NO: 5 to SEQ ID NO: 8, but is not limited thereto.

Specifically, the derivative in the present invention may be any one or more selected from the group consisting of SEQ ID NO: 1 to SEQ ID NO: 4, but is not limited thereto.

Specifically, the tumor tissue-penetrating peptide consisting of amino acid sequences of SEQ ID NO: 5 to SEQ ID NO: 8 of the present invention and a derivative thereof may have a sequence represented by the following Chemical Formula 1 and exhibit penetrability into tumor tissues, but are not limited thereto.

[X1-X2-Lys-Phe-X3-X4-Ile-Leu-X5-Tyr-Leu-X6] [Chem. 1]

In Chemical Formula 1, X1 to X6 may be selected from basic amino acids, and may be selected from arginine, lysine, leucine, or phenylalanine. Preferably, the tumor tissue-penetrating peptide consisting of any one of amino acid sequences of SEQ ID NO: 5 to SEQ ID NO: 8 and a derivative thereof may have the sequence represented by Chemical Formula 1, and the derivative of the present invention may consist of amino acid sequences of SEQ ID NO: 1 to SEQ ID NO: 4, but the peptide and derivative thereof are not limited thereto.

SEQ ID NO: 1:
H₂N-[Lys-Lys-Leu-Phe-Lys-Lys-Ile-Leu-Lys-Tyr-Leu-
Lys]-CO₂H,
SEQ ID NO: 2:
H₂N-[Arg-Arg-Leu-Phe-Arg-Arg-Ile-Leu-Arg-Tyr-Leu-
Arg]-CO₂H,
SEQ ID NO: 3:
H₂N-[Arg-Phe-Leu-Phe-Arg-Leu-Ile-Leu-Arg-Tyr-Leu-
Arg]-CO₂H,
SEQ ID NO: 4:
H₂N-[Arg-Arg-Leu-Phe-Arg-Leu-Ile-Leu-Arg-Tyr-Leu-
Phe]-CO₂H,
SEQ ID NO: 5:
H₂N-[Arg-Lys-Leu-Phe-Lys-Arg-Ile-Leu-Arg-Tyr-Leu-
Lys]-CO₂H,
SEQ ID NO: 6:
H₂N-[Lys-Arg-Leu-Phe-Arg-Lys-Ile-Leu-Lys-Tyr-Leu-
Arg]-CO₂H,
SEQ ID NO: 7:
H₂N-[Lys-Arg-Leu-Phe-Lys-Arg-Ile-Leu-Arg-Tyr-Leu-
Lys]-CO₂H,
and
SEQ ID NO: 8:
H₂N-[Arg-Lys-Leu-Phe-Arg-Lys-Ile-Leu-Lys-Tyr-Leu-
Arg]-CO₂H

Specifically, the derivative may has a substitution of an amino acid corresponding to any one position selected from the group consisting of 1st, 2nd, 5th, 6th, 9th, and 12th positions from the N-terminus of the tumor tissue-penetrating peptide consisting of any one of amino acid sequences of SEQ ID NO: 5 to SEQ ID NO: 8 with another basic amino acid.

Amino acid substitution may generally occur based on the polarity, charge, solubility, hydrophobicity, hydrophilicity and/or amphipathic nature of the residues. Specifically, among electrically charged amino acids, positively charged (basic) amino acids include arginine (R), lysine (K), and histidine (H) and negatively charged (acidic) amino acids include glutamate (E) and aspartate (D); among uncharged amino acids, nonpolar amino acids include glycine (G), alanine (A), valine (V), leucine (L), isoleucine (I), methionine (M), phenylalanine (F), tryptophan (W), and proline (P) and polar or hydrophilic amino acids include serine (S), threonine (T), cysteine (C), tyrosine (Y), asparagine (N), and glutamine (Q), and among the non-polar amino acids, aromatic amino acids include phenylalanine, tryptophan, and tyrosine.

In the present invention, ‘substitution with another basic amino acid’ is not limited as long as it is substitution with a basic amino acid that is different from the amino acid before substitution. In other words, the substitution may be substitution of an amino acid before substitution present at any one position selected from the group consisting of 1st, 2nd, 5th, 6th, 9th, and 12th positions from the N-terminus of the tumor tissue-penetrating peptide consisting of any one of amino acid sequences of SEQ ID NO: 5 to SEQ ID NO: 8 with another basic amino acid, may specifically be substitution of an amino acid present at any one position selected from the group consisting of the X1, X2, X3, X4, X5, and X6 positions with another basic amino acid. Specifically, the basic amino acid may be selected from histidine, lysine, or arginine, and may be lysine or arginine, but is not limited thereto.

For the purpose of the present invention, a derivative of the tumor tissue-penetrating peptide consisting of any one of amino acid sequences of SEQ ID NO: 5 to SEQ ID NO: 8 may be any one or more selected from the group consisting of SEQ ID NO: 1 to SEQ ID NO: 4, but is not limited thereto.

In the present invention, “fragment” refers to a partial sequence of a peptide having a specific sequence. The fragment of the present invention may be a partial sequence of a tumor tissue-penetrating peptide consisting of any one of amino acid sequences of SEQ ID NO: 5 to SEQ ID NO: 8 or a derivative thereof, and may exhibit penetrability into tumor tissues as the tumor tissue-penetrating peptide or a derivative thereof.

The tumor tissue-penetrating peptide consisting of any one of amino acid sequences of SEQ ID NO: 5 to SEQ ID NO: 8, a derivative thereof, or a fragment thereof may be derived from natural products, but is not limited thereto, and includes sequences having the same activity as the tumor tissue-penetrating peptide, a derivative thereof, or a fragment thereof without limitation.

The tumor tissue-penetrating peptide in the present invention is described as a tumor tissue-penetrating peptide consisting of any one of amino acid sequences of SEQ ID NO: 5 to SEQ ID NO: 8, a derivative thereof, or a fragment thereof, but does not exclude the meaningless addition of sequences before or after the amino acid sequence of the tumor tissue-penetrating peptide consisting of any one of amino acid sequences of SEQ ID NO: 5 to SEQ ID NO: 8, a derivative thereof, or a fragment thereof, or silent mutations that allow the tumor tissue-penetrating peptide to maintain the same function as the tumor tissue-penetrating peptide. It may be apparent to those skilled in the art that one corresponds to the tumor tissue-penetrating peptide of the present invention as long as it has the same or equivalent activity as the tumor tissue-penetrating peptide consisting of any one of amino acid sequences of SEQ ID NO: 5 to SEQ ID NO: 8, a derivative thereof, or a fragment thereof. As a specific example, the tumor tissue-penetrating peptide of the present invention may be a peptide consisting of the amino acid sequence of the tumor tissue-penetrating peptide consisting of any one of amino acid sequences of SEQ ID NO: 5 to SEQ ID NO: 8, a derivative thereof, or a fragment thereof or an amino acid sequence having 80%, 90%, 95%, 96%, 97%, 98%, or 99% or more homology or identity thereto.

In the present invention, even if it is described as ‘a peptide containing an amino acid sequence described by a specific sequence number’, ‘a peptide consisting of an amino acid sequence described by a specific sequence number’, or ‘a peptide having an amino acid sequence described by a specific sequence number’, it is obvious that a peptide having an amino acid sequence in which some sequences are deleted, modified, substituted, conservatively substituted, or added can also be used in the present invention as long as it has the same or equivalent activity as the peptide consisting of the amino acid sequence of the corresponding sequence number. Examples thereof include a case having an addition of a sequence, a naturally occurring mutation, a silent mutation, or a conservative substitution, which does not change the function of the peptide, at the N-terminus and/or C-terminus of the amino acid sequence.

The term “conservative substitution” means substituting one amino acid with another amino acid having similar structural and/or chemical properties. Such amino acid substitution may generally occur based on similarity in polarity, charge, solubility, hydrophobicity, hydrophilicity and/or amphipathic nature of the residues. Typically, conservative substitution may have little or no effect on the activity of the peptide.

The peptide of the present invention can be prepared by sequentially forming an amide bond of one or more amino acids or suitably protected amino acids to an amino acid skeleton constantly bound to the solid phase, but is not limited thereto. The peptide can undergo insertion, substitution, or deletion of other amino acids without greatly reducing the stability, and this also falls within the scope of the present invention.

The tumor tissue-penetrating peptide of the present invention may be prepared in the form in which a known cell permeable peptide, which promotes intracellular movement, is additionally bound to the C-terminus or N-terminus. For example, the known cell permeable peptide may be TAT peptide (Arg-Lys-Lys-Arg-Arg-Tyr-Arg-Arg-Arg) and Tat-PTD peptide (Gly-Arg-Lys-Lys-Arg-Arg-Gln-Arg-Arg-Arg: Tat PTD), but the present invention is not limited to this, and any cell permeable peptide known in the art can be used as long as it does not inhibit the activity of the present invention.

The peptides and compounds of the present invention may also be prepared in the form of metal complexes, the metal may be selected from copper, magnesium, calcium, iron, zinc, nickel, silver, germanium, or gallium, but is not limited thereto, and copper may be preferably used, but the metal is not limited thereto.

Meanwhile, the peptide of the present invention may exist in salt form. The salt form usable in the present invention may be those formed during final separation and purification of the compound or by the reaction of an amino group with an appropriate acid. For example, the acid addition salt may be an acetate, an adipate, an alginate, a citrate, an aspartate, a benzoate, a benzenesulfonate, a bisulfate, a butyrate, a camphorate, a camphorsulfonate, a digluconate, a glycerophosphate, a hemisulfate, a heptanoate, a hexanoate, a formate, a fumarate, a hydrochloride, a hydrobromide, a hydroiodide, a 2-hydroxyethanesulfonate, a lactate, a maleate, a mesitylene sulfonate, a methanesulfonate, a naphthylene sulfonate, a nicotinate, a 2-naphthalene sulfonate, an oxalate, a pamoate, a pectinate, a persulfate, a 3-phenylpropionate, a picrate, a pivalate, a propionate, a succinate, a tartrate, a trichloroacetate, a trifluoroacetate, a phosphate, a glutamate, a bicarbonate, a p-toluenesulfonate, and an undecanoate, but is not limited thereto. The acid that may be used to form the acid addition salt may be inorganic acids such as hydrochloric acid, hydrobromic acid, sulfuric acid, and phosphoric acid and organic acids such as oxalic acid, maleic acid, succinic acid, and citric acid, but is not limited thereto. Preferably, the peptide of the present invention can be prepared in the form of a trifluoroacetate or acetate.

The amino group or carboxyl group of the amino acid used to prepare the peptide contained in the composition of the present invention may be protected by a suitable protecting group. The protected amino acid may be attached to a solid support or reacted in a solution by adding the next amino acid under conditions suitable to form an amide bond. The protecting group may be completely removed prior to addition of the amino acid protected by a suitable protecting group. After all amino acids are linked as desired, the amino acids may be separated sequentially or simultaneously from the free residual protecting groups and the free solid support to obtain the final desired peptide.

The most preferred synthesis method for preparing the peptide compound of the present invention may be a solid-phase peptide synthesis method using a solid-phase polymer support, and the α-amino group of the peptide prepared through the method may be protected by an acid or base sensitive functional group. The protecting group of the amino acid at this time is required to have stable properties under the peptide condensation reaction conditions, and is required to have the properties of being readily removable without destruction of the extending peptide chain or without racemization of any chiral centers contained therein. Therefore, the suitable protecting groups may be 9-fluorenylmethyloxycarbonyl (Fmoc), t-butoxycarbonyl (Boc), benzyloxycarbonyl (Cbz), biphenylisopropyl-oxycarbonyl, t-amyloxycarbonyl, isobornyloxycarbonyl, (α,α)-dimethyl-3,5-dimethoxybenzyloxycarbonyl, O-nitrophenylsulfenyl, and 2-cyano-t-butyloxycarbonyl, and the like, and other suitable protecting groups known in the art for this purpose can also be used within the scope of the present invention.

The most preferred protecting group for the amino acid used in the peptide synthesis of the present invention is 9-fluorenylmethyloxycarbonyl (Fmoc) protecting group.

In particular, the protecting group for the amino acid residue used in the peptide synthesis of the present invention is preferably t-butyl (t-Bu) for N-methylglutamic acid; t-butoxycarbonyl (Boc) for lysine; t-butyl (t-Bu) for serine; t-butyl (t-Bu) for threonine and allothreonine; and trityl (Trt) for cysteine, but the present invention is not limited thereto.

In solid-phase peptide synthesis methods, the C-terminal amino acid may be attached to a suitable solid support or resin. Suitable solid supports useful for the synthesis are preferably materials that are inert to the reagents and reaction conditions of the stepwise condensation-deprotection reaction and insoluble in the medium used, and may be, for example, 2-chlorotrityl chloride resin, rink amid, or rink amid 4-methylbenzylhydrylamine resin (rink amid MBHA resin).

In particular, preferred solid supports for C-terminal peptides may be 2-chlorotrityl chloride, rink amid or rink amid 4-methylbenzylhydrylamine resin (rink amid MBHA resin) available from Novabiochem Corporation.

The C-terminal amide may be condensed (bound, coupled) to a resin or solid-phase support through condensation by activating carboxylic acid to N,N′-dicyclohexylcarbodiimide (DCC), N,N-diisopropylcarbodiimide (DIC), [0-(7-azabenzotriazol-1-yl)-1,1,3,3-tetramethyluronium hexafluorophosphate] (HATU) or O-benzotriazol-1-yl-N,N,N,N′-tetramethyluroniumhexafluorophosphate (HBTU) in a solvent such as dichloromethane, N-methylpyridone (NMP) or DMF at a temperature of 10° C. to 50° C., preferably at a temperature of 30° C. for 1 to 24 hours in the presence or absence of 4-dimethylaminopyridine (DMAP), 1-hydroxybenzotriazole (HOBt), N-methylmorpholine (NMM), benzotriazoll-yloxy-tris(dimethylamino)phosphonium-hexafluorophosphate (BOP) or bis(2-oxo-3-oxazolidinyl)phosphine chloride (BOPCl).

In a case where the solid support is rink amid 4-methylbenzylhydrylamine resin, the Fmoc functional group as a preferred protecting group is cleaved using an excess of a secondary amine solution, preferably a 20% piperidine DMF solution, before condensation with the C-terminal amino acid. Preferred reagents used to condense the desired amino acid to the deprotected 4-(2′,4′-dimethoxyphenyl-Fmoc-aminomethyl)phenoxyacetamidoethyl resin are condensation reaction reagents such as N-methylmorpholine (NMM), 1-hydroxybenzotriazole (HOSt) and O-(7-azabenzotriazol-1-yl)-1,1,3,3-tetramethyluroniumhexafluorophosphate](HATU), O-benzotriazol-1-yl-N,N,N′,N′-tetramethyluroniumhexafluorophosphate (HBTU), N,N′-dicyclohexylcarbodiimide (DCC) or N,N′-diisopropylcarbodiimide (DIC) for suitably protected amino acids in DMF solvent.

The continuous condensation of amino acids conducted in the present invention may be conducted manually or using an automatic peptide synthesizer widely known in the related art. As preferred conditions for synthesis reaction, the α-amino acid protected by the Fmoc group is deprotected through treatment with a secondary amine solution, preferably piperidine, and then washed with a sufficient excess of solvent, each of the other protected amino acids to be condensed is subsequently added in a 3- to 7-fold molar excess, and the reaction is conducted preferably in a DMF solvent.

In the final step of peptide synthesis using a solid-phase resin of the present invention, the peptide to be obtained may be removed from the resin continuously or by one operation, and the protecting groups, which protect the respective amino acid residues, may be deprotected. The conditions for removal of the peptide from the resin and deprotection of the protecting groups present on the residues are generally obtained by treating a cleavage reagent cocktail that cleaves the bond between the resin and the peptide, for example, a dichloromethane mixed cocktail solution of trifluoroacetic acid (TFA), triisopropylsilane (TIS), thioanisole, and water, or ethanedithiol (EDT). The mixed solution obtained in this way may be treated with an excess of refrigerated diethyl ether solvent to produce a precipitate. The precipitate obtained as above is centrifuged for complete precipitation, and excess trifluoroacetic acid, triisopropylsilane, thioanisole, water, and ethanedithiol are first removed, and the above procedure is repeated two or more times to obtain a solidified precipitate. At this time, the completely deprotected peptide salt may be separated and purified using a mixed solvent of water and acetonitrile solvent by reversed-phase high-performance liquid chromatography (HPLC). The separated and purified peptide solution may be completely concentrated and dried by lyophilization to obtain a solid peptide.

The present invention provides a composition for drug delivery and a composition for delivery of a physiologically active substance to tumor.

The composition for drug delivery of the present invention may be for drug delivery to tumor.

The composition for delivery to tumor of the present invention may allow a physiologically active substance to penetrate into tumor.

In the present invention, the “physiologically active substance” is a general term for substances that have physiological action in the living body. The physiologically active substance and drug may be hydrophilic, hydrophobic, or poorly soluble, and include, without limitation, natural products, chemical compounds, proteins, peptides, amino acids, nucleic acids, lipids, liposomes, and polymers as long as they can have physiological action. The physiologically active substance and drug of the present invention may be used singly, and may include, but is not limited to, combinations with other physiologically active substances or drugs, combinations with carriers, and combinations with known tumor tissue-penetrating peptides.

In the present invention, the “drug” is a general term for substances that can be applied to tumor and have beneficial effects on a subject. The drug may be included in the physiologically active substance.

As an exemplary embodiment, the drug may be a drug for tumor suppression, but is not limited thereto.

As an exemplary embodiment, the physiologically active substance or drug may include, but is not limited to, any one or more selected from the group consisting of doxorubicin, gemcitabine, taxol, and albumin.

The tumor tissue-penetrating peptide consisting of any one of amino acid sequences of SEQ ID NO: 5 to SEQ ID NO: 8, a derivative thereof or a fragment thereof may be directly or indirectly linked to a drug or physiologically active substance, but is not limited thereto.

As an exemplary embodiment, the tumor tissue-penetrating peptide consisting of any one of amino acid sequences of SEQ ID NO: 5 to SEQ ID NO: 8 of the present invention, a derivative thereof or a fragment thereof and the physiologically active substance or drug may be separated from each other or fused together, but are not limited thereto.

The tumor tissue-penetrating peptide consisting of any one of amino acid sequences of SEQ ID NO: 5 to SEQ ID NO: 8, a derivative thereof or a fragment thereof and the physiologically active substance or drug may be linked to each other directly or through a linker or may further contain other protein moieties, but are not limited thereto. As the linking method of the present invention, methods performed in the art may be used without limitation as long as they do not change the structure or activity of the linked tumor tissue-penetrating peptide consisting of any one of amino acid sequences of SEQ ID NO: 5 to SEQ ID NO: 8, a derivative thereof or a fragment thereof and physiologically active substance or drug. The linker may be a peptide linker consisting of 1 to 20 amino acids or a non-peptide linker, but is not limited thereto.

As an exemplary embodiment, the tumor tissue-penetrating peptide consisting of any one of amino acid sequences of SEQ ID NO: 5 to SEQ ID NO: 8 of the present invention, a derivative thereof, or a fragment thereof may be linked to a drug or physiologically active substance by a non-covalent bond (for example, ionic bond, hydrogen bond, or van der Waals bond), but is not limited thereto.

As an exemplary embodiment, the tumor tissue-penetrating peptide consisting of any one of amino acid sequences of SEQ ID NO: 5 to SEQ ID NO: 8 of the present invention, a derivative thereof or a fragment thereof may be linked (for example, by covalent bond) to a drug or physiologically active substance, but is not limited thereto. The tumor tissue-penetrating peptide, a derivative thereof, or a fragment thereof and a drug may be directly linked to each other or linked through a linker or may further contain other protein moieties, but are not limited thereto.

As an exemplary embodiment, the tumor tissue-penetrating peptide may undergo self-assembling or self-fusion with a drug or physiologically active substance.

As an exemplary embodiment, the tumor tissue-penetrating peptide may be one or more molecules surrounding or fused with a drug or physiologically active substance, but is not limited thereto.

The composition for drug delivery of the present invention may further contain carriers, excipients, diluents, antioxidants, and/or buffers for use, if necessary, but is not limited thereto.

As an exemplary embodiment, the carrier may be a liposome, but is not limited thereto. The liposome refers to a hollow structure formed by a phospholipid bilayer in an aqueous solution. The liposome may contain a drug in the hollow structure, but is not limited thereto.

Still another aspect of the present invention provides a carrier for drug delivery, containing a tumor tissue-penetrating peptide consisting of any one of amino acid sequences of SEQ ID NO: 5 to SEQ ID NO: 8, a derivative thereof, or a fragment thereof.

Any one of the amino acid sequences of SEQ ID NO: 5 to SEQ ID NO: 8, the tumor tissue-penetrating peptide, derivative, fragment, drug, and the like are as described above.

In the present invention, “carrier” means one that can support an arbitrary substance or component, including a composition as an example, and may be used interchangeably with a supporting body, impregnation material, or mediator. The composition, which is an example of the carrier, may be applied and delivered to the skin through an application means such as a hand, puff, tip, or brush, or may be injected into a subject.

Still another aspect of the present invention provides a pharmaceutical composition containing the composition for drug delivery or the composition for delivery of a physiologically active substance to tumor of the present invention, Any one of the amino acid sequences of SEQ ID NO: 5 to SEQ ID NO: 8, the tumor tissue-penetrating peptide, derivative, and fragment are as described above.

The pharmaceutical composition of the present invention may further contain a pharmaceutically acceptable carrier as well as contain the tumor tissue-penetrating peptide consisting of amino acid sequences of SEQ ID NO: 5 to SEQ ID NO: 8, a derivative thereof, or a fragment thereof.

In the present invention, “prevention” means any action that prevents or delays disease by administration of the composition of the present invention, and “treatment” means any action that improves or beneficially changes the symptoms of disease by administration of the composition of the present invention.

The disease may be cancer.

In the present invention, “pharmaceutically acceptable” means that the substance does not irritate the organism upon administration, does not inhibit the biological activity and properties of an administered compound, and can be commonly used in the pharmaceutical field. The pharmaceutical composition of the present invention may be formulated with a carrier and used as a food, medicine, feed additive, drinking water additive, or the like.

The type of the carrier is not particularly limited, and any carrier commonly used in the art may be used. Non-limiting examples of the carrier include saline solution, sterile water, Ringer's solution, buffered saline solution, albumin, lactose, dextrose, sucrose, sorbitol, mannitol, xylitol, erythritol, maltitol, maltodextrin, glycerol, and ethanol. These may be used singly or in a mixture of two or more thereof, but are not limited thereto.

If necessary, other pharmaceutically acceptable additives such as excipients, diluents, antioxidants, buffers, or bacteriostatic agents may be added to the pharmaceutical composition of the present invention for use, and fillers, extenders, wetting agents, disintegrants, dispersants, surfactants, binders, or lubricants may be further added to the pharmaceutical composition for use, but the pharmaceutical composition is not limited thereto.

The pharmaceutical composition of the present invention may contain a pharmaceutically effective amount of the drug. In the present invention, the term “pharmaceutically effective amount” means an amount sufficient to treat disease with a reasonable benefit/risk ratio applicable to medical treatment, and the drug may be administered generally in an amount of 0.001 to 1000 mg/kg, specifically in an amount of 0.05 to 200 mg/kg, more specifically in an amount of 0.1 to 100 mg/kg, still more specifically in an amount of 0.1 to 50 mg/kg one time a day or several times a day in divided doses. However, for the purpose of the present invention, it is preferable to apply the specific therapeutically effective amount for a specific patient differently depending on the type and degree of response to be achieved, and in some cases, various factors, including the specific composition, including whether other agents are used, the patient's age, weight, general health condition, sex and diet, administration time, administration route and the secretion rate of the composition, treatment period, and drugs used together or simultaneously with the composition, and similar factors well known in the medical field.

The pharmaceutical composition of the present invention may be administered daily or intermittently, and the number of administration times per day can be one time or two to three times. The pharmaceutical composition of the present invention may be used singly or in combination with other drug treatments. Considering all of the factors, it is important to administer the pharmaceutical composition in the minimum amount to achieve the maximum effect without side effects, and the amount may be easily determined by those skilled in the art.

In detail, solid preparations for oral administration include tablets, pills, powders, granules, capsules, and the like, and these solid preparations may be prepared by mixing the compound with at least one or more excipients, for example, starch, calcium carbonate, sucrose, lactose, and gelatin. In addition to simple excipients, lubricants such as magnesium stearate and talc may also be used. Liquid preparations for oral administration include suspensions, oral solutions, emulsions, and syrups, and may contain various excipients, for example, wetting agents, sweeteners, fragrances, and preservatives in addition to the commonly used simple diluents such as water and liquid paraffin. Preparations for parenteral administration include injections, sterile aqueous solutions, non-aqueous solvents, suspensions, emulsions, lyophilized preparations, and suppositories. As the non-aqueous solvents and suspensions, propylene glycol, polyethylene glycol, vegetable oils such as olive oil, injectable esters such as ethyl oleate, and the like may be used. As a base for suppositories, Witepsol, Macrogol, Tween 61, cacao, laurin, glycerogelatin, and the like may be used.

Non-limiting examples of the skin external preparations include aerosols, sprays, cleansers, ointments, application powders, oils, and creams, but are not limited thereto as long as it can function as a skin external preparation. In order to formulate the pharmaceutical composition of the present into a skin external preparation, sterile aqueous solutions, non-aqueous solvents, suspensions, emulsions, lyophilized preparations, external preparations, and the like may be used. As the non-aqueous solvents and suspensions, propylene glycol, polyethylene glycol, vegetable oils such as olive oil, injectable esters such as ethyl oleate, and the like may be used, and the non-aqueous solvents and suspensions are not limited thereto.

More specifically, in the case of formulating the pharmaceutical composition of the present invention, the pharmaceutical composition of the present invention may be mixed with a stabilizer or buffer in water to prepare a solution or suspension, and this may be formulated in units such as ampoules. In the case of formulating the pharmaceutical composition of the present invention into an aerosol, propellants, and the like may be mixed with additives to disperse the water-dispersed concentrate or wet powder, but the pharmaceutical composition is not limited thereto.

In the case of formulating the pharmaceutical composition of the present invention into an ointment, cream, or the like, the pharmaceutical composition may be formulated using animal oil, vegetable oil, wax, paraffin, starch, tragacanth, cellulose derivatives, polyethylene glycol, silicone, bentonite, silica, talc, zinc oxide, and the like as a carrier, but is not limited thereto.

The pharmaceutical composition of the present invention may be formulated into a skin external preparation, more preferably may be a preparation selected from the group consisting of gels, patches, sprays, ointments, plasters, lotions, liniments, pastes, and cataplasmas, but is not limited thereto.

The pharmaceutically effective amount of the pharmaceutical composition of the present invention may vary depending on the formulation method, administration method, administration time, and/or administration route of the pharmaceutical composition, and may vary depending on various factors, including the type and degree of response to be achieved by administration of the pharmaceutical composition, the type, age, weight, general health condition of the subject to be administered, symptoms and severity of the disease, sex, diet, excretion, drugs used simultaneously or at different times for the subject, and other components of the composition, and similar factors well known in the pharmaceutical field. Those skilled in the art can easily determine and prescribe an effective dosage for the desired treatment. The pharmaceutical composition of the present invention may be administered once a day or several times a day in divided doses. Therefore, the dosage does not limit the scope of the present invention in any way.

In the present invention, administration may be as an external preparation, and the preferred dosage of the pharmaceutical composition of the present invention may be 1 mg/kg to 1000 mg/kg per day.

In the present invention, “administration” means introducing the composition of the present invention into a subject by any suitable method, and the composition may be administered through various oral or parenteral routes. Specifically, for parenteral administration, injection methods such as nasal administration, external application to the skin, intraperitoneal injection, rectal injection, subcutaneous injection, intravenous injection, intramuscular injection or intrathoracic injection may be selected. The composition may be administered in a pharmaceutically effective amount.

The administration route and administration method of the pharmaceutical composition of the present invention may each be independent, there is no particular limitation on the method, and any administration route and any administration method may be adopted as long as the pharmaceutical composition can reach the desired site. The pharmaceutical composition may be administered by injection or external application to the skin, but is not limited thereto.

The pharmaceutical composition of the present invention may be administered by intravenous injection, intramuscular injection, application, or spraying, but is not limited thereto.

As an exemplary embodiment, the pharmaceutical composition of the present invention may further contain a drug, and the drug may be a drug for tumor suppression (anticancer agent), but is not limited thereto.

As an exemplary embodiment, the pharmaceutical composition of the present invention may be for prevention or treatment of cancer or angiogenesis-related diseases, but is not limited thereto.

In the present invention, “cancer” refers to the presence of cells with characteristics of cancer-causing cells, such as uncontrolled proliferation, immortality, metastatic potential, rapid growth and proliferation rates, and characteristic morphological properties known in the art. The cancer may be used in the same sense as “tumor.” The cancer may be a solid cancer, may be colorectal cancer, breast cancer, prostate cancer, lung cancer, small cell lung cancer, non-small cell lung cancer, colon cancer, bone cancer, pancreatic cancer, gallbladder cancer, skin cancer, head or neck cancer, skin or intraocular melanoma, uterine cancer, ovarian cancer, rectal cancer, stomach cancer, anal cancer, colon cancer, fallopian tube carcinoma, endometrial carcinoma, cervical carcinoma, vaginal carcinoma, vulvar carcinoma, esophageal cancer, small intestine cancer, endocrine cancer, thyroid cancer, parathyroid cancer, adrenal cancer, soft tissue sarcoma, urethral cancer, penile cancer, chronic or acute leukemia, lymphocytic lymphoma, bladder cancer, kidney or ureteral cancer, renal cell carcinoma, renal pelvic carcinoma, central nervous system (CNS) tumor, primary central nervous system lymphoma, spinal cord tumor, brainstem glioma, pituitary adenoma, liver cancer, salivary gland cancer, and the like, more specifically may be any one or more selected from the group consisting of pancreatic cancer, gallbladder cancer, kidney cancer, colorectal cancer, lung cancer, liver cancer, skin cancer, breast cancer, bladder cancer, and stomach cancer, but is not limited thereto. The cancer may include malignant cancer as well as pre-malignant cancer.

In particular, therapeutic agents for solid cancers have a relatively low response rate compared to that of therapeutic agents for blood cancer, this makes it more difficult to treat solid cancers, and one of the reasons for this is the physiological properties of tumor tissues. Physiological factors that interfere with the penetration and distribution of antibodies into tumor tissues may be classified into four: endothelial barrier, high interstitial fluid pressure in tumor tissues, stromal impediment, and epithelial barrier.

Among these, the stromal impediment is an extracellular matrix barrier encountered when antibodies escape into microvessels and are convected to tissues, and is mainly composed of collagen and hyaluronan, and depending on the composition thereof, there is a site where the drug is well distributed and a site where the drug is not well distributed, causing uneven drug distribution. When the expression level of extracellular matrix increases, cell density increases by solid stress, making drug delivery more difficult. On the other hand, the composition for drug delivery and the composition for delivery of a physiologically active substance to tumor of the present invention can deliver drugs or physiologically active substances to solid cancers as well.

In the present invention, the angiogenesis-related disease may be one or more selected from the group consisting of diabetic retinopathy, macular degeneration, age-related macular degeneration, retinopathy of prematurity, corneal transplant rejection, neovascular glaucoma and posterior phakic fibroplasia, epidemic keratoconjunctivitis, vitamin A deficiency, excessive wearing of contact lenses, atopic keratitis, superior limbal keratitis, pterygium psoriatic keratitis, Sjögren's syndrome, erythematous acne, phlyctenotic keratoconjunctivitis, syphilis, mycobacterial infection, steatorrhea, chemical burns, bacterial ulcers, fungal ulcers, herpes simplex infection, herpes zoster infection, protozoal infection, Kaposi's sarcoma, Mooren's ulcer, Therrien's marginal corneal degeneration, peripheral keratolysis, mental trauma, rheumatoid arthritis, systemic erythema, polyarteritis, Wegener's sarcoidosis, scleritis, Steve Johnson's disease, peripheral scarring radial keratotomy, and corneal transplant rejection, but is not limited thereto.

Still another aspect of the present invention provides a food composition containing the composition for drug delivery or the composition for delivery of a physiologically active substance to tumor of the present invention.

The composition for drug delivery, physiologically active substance, composition for delivery to tumor, and the like are as described above.

The term “food” in the present invention includes meat, sausages, bread, chocolate, candy, snacks, confectionery, pizza, ramen, other noodles, gum, dairy products including ice cream, various soups, beverages, tea, drinks, alcoholic beverages, vitamin complexes, health functional foods, and health foods, and includes all foods in the conventional sense.

The health functional food is the same term as food for special health use (FoSHU), and refers to a food with high medical and health effects that has been processed so as to efficiently exhibit bioregulatory functions in addition to nutrition supply. Here, “function(al)” means adjusting nutrients for the structure and function of the human body or obtaining useful effects for health purposes, such as physiological action. The food of the present invention can be prepared by methods commonly used in the industry, and at the time of preparation, raw materials and ingredients commonly added in the industry may be added. The food may also be prepared in any formulation without limitation as long as it is a formulation recognized as a food. The food composition of the present invention may be prepared in various formulations, and unlike general medicines, the food composition is prepared using foods as raw materials, thus has the advantage of not having side effects that may occur when a medicine is taken for a long period of time, and is highly portable, so the food of the present invention can be consumed as a supplement.

The health food refers to a food that has a positive health maintenance or promotion effect compared to a general food, and the health supplement food refers to a food for health supplement purposes. In some cases, the terms health functional food, health food, and health supplement food are used interchangeably.

Specifically, the health functional food is a food prepared by adding the composition of the present invention to food materials, such as beverages, teas, spices, gum, and confectionery, or by encapsulating, powdering, suspending, or the like the composition, and brings about a specific health effect when consumed, and unlike general medicines, the health functional food is prepared using foods as raw materials and thus has the advantage of not having side effects that may occur when a medicine is taken for a long period of time.

The food composition of the present invention is greatly useful since the food composition can be consumed on a daily basis and expected to have a high disease preventing or improving effect.

The composition may further contain a physiologically acceptable carrier, but the type of carrier is not particularly limited, and any carrier commonly used in the art may be used.

The composition may contain additional ingredients that are commonly used in food compositions and can improve odor, taste, appearance, and the like.

The composition may contain, for example, vitamins A, C, D, E, B1, B2, B6, and B12, niacin, biotin, folate, and pantothenic acid. The composition may also contain minerals such as zinc (Zn), iron (Fe), calcium (Ca), chromium (Cr), magnesium (Mg), manganese (Mn), copper (Cu), and chromium (Cr). The composition may also contain amino acids such as lysine, tryptophan, cysteine, and valine.

The composition may contain food additives such as preservatives (potassium sorbate, sodium benzoate, salicylic acid, sodium dehydroacetate, and the like), disinfectants (bleaching powder, high bleaching powder, sodium hypochlorite, and the like), antioxidants (butylhydroxyanisole (BHA), butylhydroxytoluene (BHT), and the like), colorants (tar color and the like), coloring agents (sodium nitrite, nitrous acid sodium salt, and the like), bleaching agents (sodium sulfite), seasonings (MSG monosodium glutamate, and the like), sweeteners (dulcin, cyclemate, saccharin, sodium, and the like), flavoring agents (vanillin, lactones, and the like), leavening agents (alum, D-potassium bitartrate, and the like), strengthening agents, emulsifiers, thickeners (starch adhesive), coating agents, gum base agents, antifoaming agents, solvents, and improvers. The additives may be selected depending on the type of food and used in an appropriate amount.

The composition of the present invention may be added as it is or used with other foods or food ingredients, and may be used appropriately according to conventional methods. The amount of the active ingredient mixed may be appropriately determined depending on the purpose of use (prevention, health, or therapeutic treatment). In general, when a food or beverage is prepared, the food composition of the present invention may be added in an amount of 50 parts by weight or less, specifically 20 parts by weight or less with respect to the food or beverage. However, the food composition may be contained in a content equal to or less than the above range when consumed for a long period of time for health and hygiene purposes. Since there is no problem in terms of safety, the active ingredient may be used in an amount exceeding the above range.

As an example of the food composition of the present invention, the food composition may be used as a health beverage composition, and in this case, the food composition may contain various flavors or natural carbohydrates as additional ingredients like ordinary beverages. The above-mentioned natural carbohydrates may be monosaccharides such as glucose and fructose; disaccharides such as maltose and sucrose; polysaccharides such as dextrins and cyclodextrins; and sugar alcohols such as xylitol, sorbitol, and erythritol. As sweeteners, natural sweeteners such as thaumatin and stevia extract; and synthetic sweeteners such as saccharin and aspartame may be used. The ratio of the natural carbohydrate is generally about 0.01 to 0.04 g, specifically about 0.02 to 0.03 g per 100 mL of the composition of the present invention.

In addition to the above, the health beverage composition may contain various nutrients, vitamins, electrolytes, flavoring agents, colorants, pectic acid, salts of pectic acid, alginic acid, salts of alginic acid, organic acids, protective colloidal thickeners, pH adjusters, stabilizers, preservatives, glycerin, alcohols, or carbonation agents. In addition to these, the health beverage composition may contain pulp for the preparation of natural fruit juice, fruit juice beverage, or vegetable beverage. These ingredients may be used independently or in a mixture. The ratio of these additives is not very important, but is generally selected in the range of 0.01 to 0.1 parts by weight per 100 parts by weight of the composition of the present invention.

The food composition of the present invention may be contained at various percentages by weight, but specifically may be contained at 0.00001% to 100% by weight or 0.01% to 80% by weight with respect to the total weight of the food composition.

As an exemplary embodiment, the food composition may be for prevention or improvement of cancer or angiogenesis-related diseases, but is not limited thereto.

The prevention is as described above.

In the present invention, “improvement” means any action that improves or beneficially changes disease by administration of the composition of the present invention.

Still another aspect of the present invention provides a feed composition containing the composition for drug delivery or the composition for delivery of a physiologically active substance to tumor of the present invention.

The feed composition may contain known carriers, stabilizers, or additives acceptable for feed in addition to the composition for drug delivery or the composition for delivery of a physiologically active substance to tumor. Examples thereof include binders, emulsifiers, and preservatives that are added to prevent deterioration in quality, and include amino acid preparations, vitamin preparations, enzyme preparations, flavors, non-protein nitrogen compounds, silicate agents, buffers, extractants, and oligosaccharides that are added to feed to increase utility. In addition to these, the feed composition may additionally contain feed mixtures and the like, but is not limited thereto. The feed composition may contain various nutrients such as vitamins, amino acids, and minerals, antioxidants, and other additives, if necessary, and may be in an appropriate form such as powder, granules, pellets, or suspensions. The feed composition of the present invention may be supplied to unitary animals singly or in mixture with feed. The feed of the present invention is not particularly limited, and may be any feed such as powdered feed, solid feed, dry feed, wet feed, moist pellet feed, dry pellet feed, EP (extruder pellet) feed, or raw food.

Still another aspect of the present invention provides a quasi-drug composition containing the composition for drug delivery or the composition for delivery of a physiologically active substance to tumor of the present invention.

The composition for drug delivery, physiologically active substance, composition for delivery to tumor, and the like are as described above.

In the present invention, the “quasi-drug” refers to articles that have a milder effect than pharmaceuticals among articles used for the purpose of diagnosing, treating, improving, alleviating, healing or preventing diseases in humans or animals. For example, according to the Pharmaceutical Affairs Act, quasi-drugs include products used to treat or prevent diseases in humans and animals and products that have a mild effect or no direct effect on the human body, excluding articles used for medicinal purposes.

The quasi-drug composition of the present invention may be prepared as an article selected from the group consisting of body cleansers, foams, soaps, masks, ointments, creams, lotions, essences, and sprays, but is not limited thereto.

In the quasi-drug composition of the present invention, the tumor tissue-penetrating peptide consisting of any one of amino acid sequences of SEQ ID NO: 5 to SEQ ID NO: 8, a derivative thereof, or a fragment thereof may be contained at 0.01% to 100.0% by weight, specifically 0.1% to 10% by weight in the total composition.

As an exemplary embodiment, the quasi-drug composition may be for prevention or improvement of cancer or angiogenesis-related diseases, but is not limited thereto.

The prevention and improvement are as described above.

Still another aspect of the present invention provides a composition for diagnosis of cancer or angiogenesis-related diseases, containing a tumor tissue-penetrating peptide consisting of any one of amino acid sequences of SEQ ID NO: 5 to SEQ ID NO: 8, a derivative thereof, or a fragment thereof.

The tumor tissue-penetrating peptide consisting of any one of amino acid sequences of SEQ ID NO: 5 to SEQ ID NO: 8 of the present invention, a derivative thereof, or a fragment thereof is selective for cancer, thus the composition may contain a means for detecting the tumor tissue-penetrating peptide consisting of any one of amino acid sequences of SEQ ID NO: 5 to SEQ ID NO: 8, a derivative thereof, or a fragment thereof, and for example, the tumor tissue-penetrating peptide consisting of any one of amino acid sequences of SEQ ID NO: 5 to SEQ ID NO: 8, a derivative thereof, or a fragment thereof may be linked to a luminescent protein. The type of luminescent protein is not limited as long as it emits light at a wavelength that can be detected by an instrument capable of detecting light. The luminescent protein may be a green fluorescent protein, a red fluorescent protein, a blue fluorescent protein, a yellow fluorescent protein, a near-infrared fluorescent protein, or a luciferase derived from prokaryotic or eukaryotic organisms. The luciferase may be a bacterial luciferase, a firefly (Photinus pyralis) luciferase, a sea pansy (Renilla) luciferase, or a Metridia luciferase.

In the present invention, the term “diagnosis” refers to the process of examining the presence or characteristics of a pathological condition. In the present invention, the diagnosis may be interpreted as examining the progress or onset of cancer or angiogenesis-related diseases.

The composition for diagnosis of the present invention may be used to determine onset of cancer at a specific location (tissue) of a subject by administering the composition for diagnosis to the subject for whom onset of cancer or an angiogenesis-related disease is to be determined and measuring the level of presence of the tumor tissue-penetrating peptide consisting of any one of amino acid sequences of SEQ ID NO: 5 to SEQ ID NO: 8, a derivative thereof, or a fragment thereof. It is obvious that as long as cancer or angiogenesis-related diseases can be diagnosed using the composition for diagnosis of the present invention, diagnosis is included within the scope of the present invention.

Still another aspect of the present invention provides a method for preventing or treating cancer or angiogenesis-related diseases, which includes administering the pharmaceutical composition to a subject other than a human. The pharmaceutical composition of the present invention, cancer, prevention, treatment, and administration are as described above.

In the present invention, the term “subject” refers to all animals, including humans, such as rats, mice, dogs, cats, cows, horses, and livestock, that are or are possibly in need of prevention or treatment of cancer or angiogenesis-related diseases. The subject may specifically be mammals, including humans.

Still another aspect of the present invention provides a use of a tumor tissue-penetrating peptide consisting of any one of amino acid sequences of SEQ ID NO: 5 to SEQ ID NO: 8, a derivative thereof, or a fragment thereof for drug delivery.

Still another aspect of the present invention provides a use of a tumor tissue-penetrating peptide consisting of any one of amino acid sequences of SEQ ID NO: 5 to SEQ ID NO: 8, a derivative thereof, or a fragment thereof for delivery of a physiologically active substance to tumor.

Still another aspect of the present invention provides a use of a composition containing the composition for drug delivery of the present invention and a drug for prevention or treatment of cancer.

Still another aspect of the present invention provides a use of a composition containing the composition for drug delivery of the present invention and a drug for prevention or treatment of an angiogenesis-related disease.

Still another aspect of the present invention provides a use of a tumor tissue-penetrating peptide consisting of any one of amino acid sequences of SEQ ID NO: 5 to SEQ ID NO: 8, a derivative thereof, or a fragment thereof for diagnosis of cancer.

Still another aspect of the present invention provides a use of a tumor tissue-penetrating peptide consisting of any one of amino acid sequences of SEQ ID NO: 5 to SEQ ID NO: 8, a derivative thereof, or a fragment thereof for diagnosis of an angiogenesis-related disease.

The tumor tissue-penetrating peptide, derivative, physiologically active substance, cancer, prevention, treatment, angiogenesis-related disease, and diagnosis are as described above.

### DETAILED DESCRIPTION OF THE INVENTION

Hereinafter, the present invention will be described in more detail with reference to Examples. However, these Examples and Experimental Examples are for illustrative purposes only and the scope of the present invention is not limited to these Examples and Experimental Examples.

### Preparation Example 1: Preparation of Tumor Tissue-Penetrating Peptide

In order to examine the function of tumor tissue-penetrating peptides, peptides of SEQ ID NO: 5 to SEQ ID NO: 8 and derivative peptides thereof were prepared.

### 1-1: Synthesis of Peptide of SEQ ID NO: 1

Measured was 71.4 mg (0.10 mmol) of 2-chlorotrityl chloride resin (1.4 mmol/g loaded resin) purchased from Novabiochem corporation, the resin was solvated with 7.5 mL of methyl chloride and reacted for 5 minutes, and then methyl chloride was removed.

Afterwards, Fmoc-Lys(Boc)-OH (281.1 mg, 0.60 mmol) was completely dissolved in 7.5 mL of MC solvent, then 0.21 mL of 1.2 mmol N,N′-diisopropylethylamine (DIPEA) was added, and the mixture was added to the resin. The reaction solution was shaken at room temperature for 12 hours and then washed with 10 mL of DMF solvent four times.

Next, 7.5 mL of 20% (w/v) piperidine DMF solution was added, and shaking was performed for 15 minutes to completely remove the 9-fluorenylmethyloxycarbonyl (Fmoc) protecting group from the substance added to the above-mentioned resin, and washing was performed with 10 mL of DMF solvent four times (with 10 mL each time, four times in total). At this stage, whether the deprotection reaction of the Fmoc protecting group proceeded was examined by conducting the Kaiser test [E. Kaiser et al. Anal. Biochem., 1970, 34(2), 595-598.].

Meanwhile, Fmoc-Leu-OH (212.0 mg, 0.60 mmol) and hexafluorophosphate benzotriazole tetramethyluronium (HBTU) (227.6 mg, 0.60 mmol) and hydroxybenzotriazole (HOBt) (81.0 mg, 0.6 mmol) were completely dissolved in 7.5 mL of DMF solvent, then DIPEA (0.21 mL, 1.2 mmol) was added, and the mixture was added to the above-mentioned resin. The reaction solution was shaken at room temperature for three hours and then washed with 10 mL of DMF solvent four times. At this stage, the Kaiser test was conducted to examine the completion of the reaction.

Next, peptides were continuously condensed (coupled) according to the same synthesis cycle as follows.
(1) Washing with DMF solvent (10 mL) four times;
(2) deprotection using 20% (w/v) piperidine DMF solution (7.5 mL) for 15 minutes;
(3) washing with DMF solvent (10 mL) four times;
(4) addition of Fmoc-amino acid, HBTU, HOBt, and DIPEA;
(5) addition of HBTU, HOBt, and DIPEA condensation reagents, amino acid activation and condensation for three hours;
(6) washing with DMF solvent (10 mL) four times;

The (1) to (6) above were repeated continuously, and at this time, the Fmoc-protected amino acid (0.60 mmol) after Fmoc-Leu-OH was added to the resin reaction vessel and condensed in the order described below.
(i) Fmoc-Tyr(tBu)-OH;
(ii) Fmoc-Lys(Boc)-OH;
(iii) Fmoc-Leu-OH;
(iv) Fmoc-Ile-OH;
(v) Fmoc-Lys(Boc)-OH;
(vi) Fmoc-Lys(Boc)-OH;
(vii) Fmoc-Phe-OH;
(viii) Fmoc-Leu-OH;
(ix) Fmoc-Lys(Boc)-OH;
(x) Fmoc-Lys(Boc)-OH;

After (6), following Fmoc-Lys(Boc)-OH condensation, finally, treatment with 20% piperidine DMF solution (7.5 mL) was performed, and then washing was performed with DMF solvent three times, MC solvent three times, and diethyl ether solvent three times, using 10 mL of the solvent each time.

Immediately after completion of the synthesis, the peptide was cleaved from the resin to which the peptide was condensed for three hours using a mixture of trifluoroacetic acid/triisopropylsilane/water (95:2.5:2.5) (10 mL), and the mixed solution thus obtained was treated with 100 mL of refrigerated diethyl ether solvent to produce a precipitate. The obtained precipitate was centrifuged for complete precipitation, and trifluoroacetic acid was first removed. The above procedure (step of adding 100 mL of diethyl ether solvent to wash the precipitate and centrifuging the precipitate-operation to remove trifluoroacetic acid that was attempted to be removed first) was repeated two times to obtain a solidified precipitate.

The precipitate (peptide) was purified over 50 minutes using a C-18 column by HPLC using a gradient solvent system of 5% to 100% acetonitrile/water containing 0.001% trifluoroacetic acid. The purified pure fraction was lyophilized to obtain 155 mg of the tumor-penetrating peptide of SEQ ID NO: 1 as a trifluoroacetate salt in white powder form.

SEQ ID NO: 1:
H₂N-[Lys-Lys-Leu-Phe-Lys-Lys-Ile-Leu-Lys-Tyr-Leu-
Lys]-CO₂H
MS (ESI) m/e, [M + H]+ = 1550.17; (155 mg)

### 1-2: Synthesis of SEQ ID NO: 2 to SEQ ID NO: 8

Peptides of SEQ ID NO: 2 to SEQ ID NO: 8 below were prepared by the same preparation process as in Preparation Example 1-1 except that the order of amino acids protected by Fmoc was changed.

SEQ ID NO: 2:
H₂N-[Arg-Arg-Leu-Phe-Arg-Arg-Ile-Leu-Arg-Tyr-Leu-
Arg]-CO₂H
MS (ESI) m/e, [M + H]+ = 1718.23; (171 mg)
SEQ ID NO: 3:
H₂N-[Arg-Phe-Leu-Phe-Arg-Leu-Ile-Leu-Arg-Tyr-Leu-
Arg]-CO₂H
MS (ESI) m/e, [M + H]+ = 1666.18; (166 mg)
SEQ ID NO: 4:
H₂N-[Arg-Arg-Leu-Phe-Arg-Leu-Ile-Leu-Arg-Tyr-Leu-
Phe]-CO₂H
MS (ESI) m/e, [M + H]+ = 1666.18; (166 mg)
SEQ ID NO: 5:
H₂N-[Arg-Lys-Leu-Phe-Lys-Arg-Ile-Leu-Arg-Tyr-Leu-
Lys]-CO₂H
MS (ESI) m/e, [M + H]+ = 1634.2; (153 mg)
SEQ ID NO: 6:
H₂N-[Lys-Arg-Leu-Phe-Arg-Lys-Ile-Leu-Lys-Tyr-Leu-
Arg]-CO₂H
MS (ESI) m/e, [M + H]+ = 1634.2; (143 mg)
SEQ ID NO: 7:
H₂N-[Lys-Arg-Leu-Phe-Lys-Arg-Ile-Leu-Arg-Tyr-Leu-
Lys]-CO₂H
MS (ESI) m/e, [M + H]+ = 1634.2; (132 mg)
SEQ ID NO: 8:
H₂N-[Arg-Lys-Leu-Phe-Arg-Lys-Ile-Leu-Lys-Tyr-Leu-
Arg]-CO₂H
MS (ESI) m/e, [M + H]+ = 1634.2; (172 mg)

### Example 1: Evaluation of Improvement in Anticancer Efficacy of Physiologically Active Substance by SEQ ID NO: 5 to SEQ ID NO: 8 and Derivatives Thereof Through In Vitro Experiment

In order to examine the function of the present invention as a tumor tissue-penetrating peptide, the effect of the present invention on improving the anticancer efficacy of doxorubicin was evaluated. Specifically, the effect of a tumor tissue-penetrating peptide consisting of any one amino acid sequence selected from SEQ ID NO: 5 to SEQ ID NO: 8 of the present invention and SEQ ID NO: 1 to SEQ ID NO: 4, derivatives thereof, on improving the anticancer efficacy of doxorubicin was evaluated.

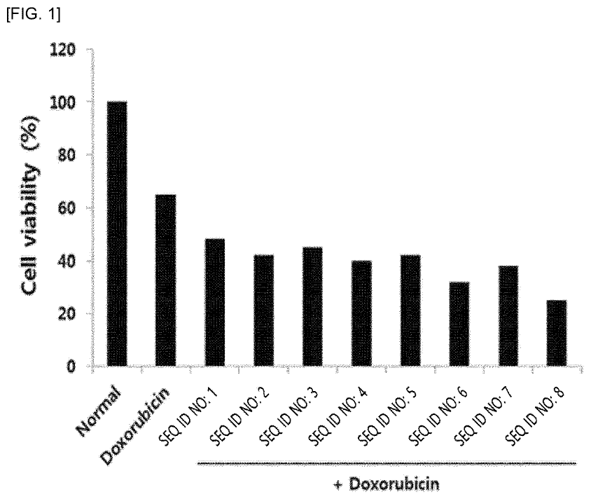

Specifically, in order to evaluate the effect of improving anticancer efficacy, AsPC-1 cells were used to perform the MTT assay, and cell viability was analyzed and compared (FIG. 1).

From the above, it has been found that SEQ ID NO: 1 to SEQ ID NO: 8 have a significant effect of improving anticancer efficacy compared to the group treated with only doxorubicin.

### MTT Assay

Pancreatic cancer cells (AsPC-1) were cultured in 96-well plates at 2×10⁵ cells/ml for 24 hours, then treated with samples at various concentrations, and cultured for another 48 hours. The cultured medium was removed, and the cells were treated with MTT solution (0.5 mg/ml in PBS). After 4 hours, the MTT solution was removed, DMSO was added to each well to dissolve formazan at 37° C. for 30 minutes, and then the absorbance was measured at 570 nm using a microplate reader (Molecular Devices Spectra MAX, Sunnyvale, CA, USA). In all experiments, the results were statistically processed by taking the average value for three wells for each concentration, and expressed as a relative viability (%) compared to that in the group treated with no sample.

### Example 2: Evaluation of NRP-1 Binding Ability of SEQ ID NO: 5 to SEQ ID NO: 8 and Derivatives Thereof

In order to examine the tumor-selective function of tumor tissue-penetrating peptides of the present invention, the binding ability of the peptides to NRP-1 was evaluated. Specifically, the binding ability of a tumor tissue-penetrating peptides consisting of any one amino acid sequence selected from SEQ ID NO: 5 to SEQ ID NO: 8 of the present invention and SEQ ID NO: 1 to SEQ ID NO: 4, derivatives thereof, to NRP-1 was evaluated.

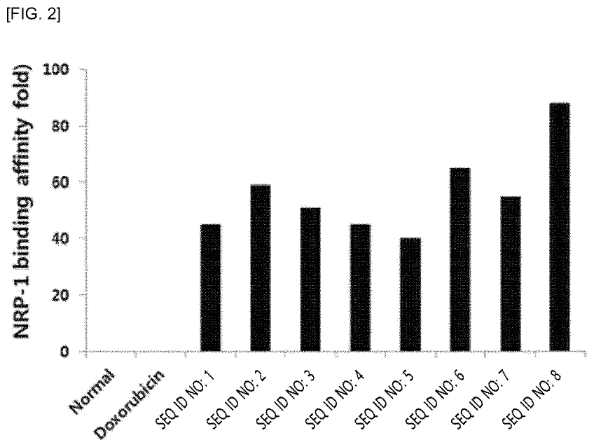

Specifically, in order to evaluate the binding ability to NRP-1, the binding ability was measured by a saturation ELISA binding assay, and compared after measurement using an ELISA plate reader (FIG. 2).

From the above, it has been found that SEQ ID NO: 1 to SEQ ID NO: 8 have binding ability to NRP-1 and thus have a tumor-selective effect.

### Saturation ELISA Binding Assay

96-well plates were coated with recombinant NRP-1 antibody and left overnight at 4° C. Each well was washed with washing buffer solution three times and then blocked with 5% BSA solution. This was left at room temperature for two hours, washed with washing buffer solution three times, and then treated with biotin-conjugated tumor tissue-penetrating peptides. After two hours, this was washed three times, then treated with 100 μL of avidin-HRP conjugated antibody, left at room temperature for one hour, and then washed again three times. TMB substrate was dispensed thereinto by 100 μL, this was left in the dark for 30 minutes, and then treated with 50 μL of stop solution, and then the absorbance was measured at 450 nm using a microspectrophotometer (Molecular Device, Sunnyvale, CA, USA).

### Example 3: Evaluation of Improvement in Anticancer Efficacy of Physiologically Active Substance by SEQ ID NO: 5 to SEQ ID NO: 8 and Derivatives Thereof Through. In Vivo Experiment

The improvement in anticancer efficacy by the tumor tissue-penetrating peptide of SEQ ID NO. 8, which was the most effective among the above-mentioned peptides of SEQ ID NO: 1 to SEQ ID NO: 8, was examined through an animal experiment.

In order to closely examine the influence of the tumor tissue-penetrating peptide of the present invention on the anticancer efficacy improving effect, the anticancer effect was examined using the AsPC-1 xenograft mouse animal model.

The experimental animals used were 6-week-old nude mice. Nude mice were raised in a constant environment of temperature, 50% humidity, and 12-hour photoperiod, and were provided with free access to food and drinking water during the experiment period. After stabilization for one week, 4×10⁶/100 μL of the prepared pancreatic cancer cells was mixed with 100 μL of Matrigel and subcutaneously injected into the mice using a syringe to induce tumor in the mice. About 10 days after injection, when the tumor grew to a certain size, each group was assigned according to tumor size, creating a tumor-induced group, a group treated with only anticancer agent (gemcitabine+taxol), a group treated with only peptide, and a group treated with anticancer agent mixed with peptide. Each drug was injected intraperitoneally a total of four times at intervals of once every two weeks, and the size of the tumor was measured every week. At the time of dissection at five weeks after tumor injection, the tumor was removed from each group and the size of the tumor was measured. The horizontal and vertical lengths of the tumor were measured, and then the size of the tumor was calculated according to the equation V=(a×b2)/2 (a: length of long part, b: length of short part).

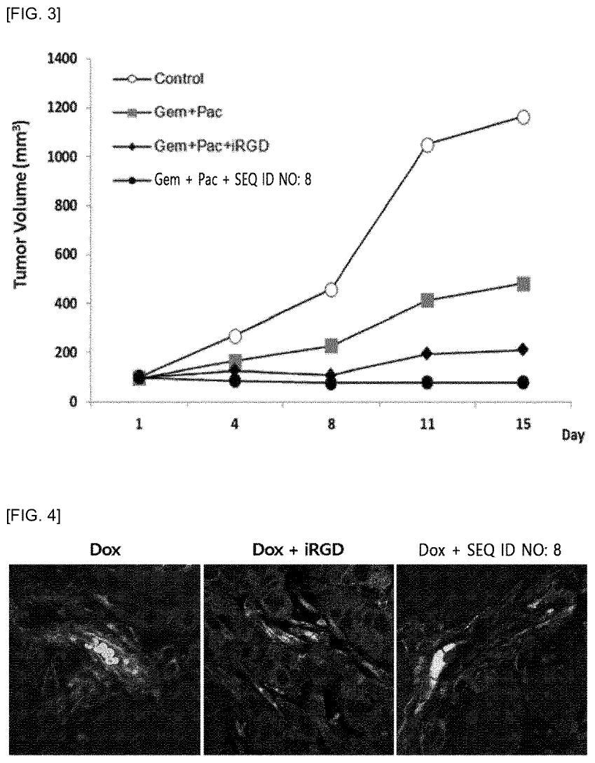

As a result, it has been found that the anticancer effect in the group treated with an anticancer agent mixed with a peptide is improved compared to that in the group administered with only an anticancer agent in vivo as well (FIG. 3).

### Example 4: Effect of Tumor Tissue-Penetrating Peptide on Improving Tumor Tissue-Penetrating Ability of Physiologically Active Substance

In order to examine the effect of the peptide of SEQ ID NO: 8 of the present invention on improving the tumor tissue-penetrating ability of a physiologically active substance, tumor-induced AsPC-1 xenograft mice were treated with only doxorubicin, doxorubicin and iRGD as a positive control, or with tumor tissue-penetrating peptide, and the effect was compared.

Specifically, iRGD or tumor tissue-penetrating peptide (5 μM/kg) was injected intravenously (iv) into AsPC-1 xenograft mice, doxorubicin (Dox; 10 mg/kg) was injected intravenously 10 minutes later, and then the tumor was extracted 24 hours later to construct paraffin slides. The slides were stained with anti-CD31-FITC (vascular endothelial cell marker), and Dox (red)/CD31 (FITC) was analyzed using a confocal microscope.

As a result, it has been found that when the mice were treated with doxorubicin and the peptide of SEQ ID NO: 8 together, the tumor tissue-penetrating ability of doxorubicin is significantly superior to that in the group treated with only doxorubicin and the group treated with doxorubicin and iRGD together (FIG. 4).

### Example 5: Effect of Tumor Tissue-Penetrating Peptide on Improving Tumor Tissue-Penetrating Ability of Various Types of Physiologically Active Substances

In order to examine the effect of the peptide of SEQ ID NO: 8 of the present invention on improving the tumor tissue-penetrating ability of physiologically active substances, FITC-albumin was injected intravenously (iv) singly or in conjugation/combination with the tumor tissue-penetrating peptide (five minutes) into tumor-induced AsPC-1 xenograft mice, the tumor was extracted 1, 3, and 24 hours later, and analysis was performed using a confocal microscope (albumin molecular weight: about 70 kDa). As a result, it was found that the tumor-penetrating ability of albumin was increased in the groups treated with albumin in conjugation/combination with the tumor tissue-penetrating peptide, and it has been thus found that the peptide of the present invention is capable of delivering a large drug having a molecular weight of about 70 kDa.

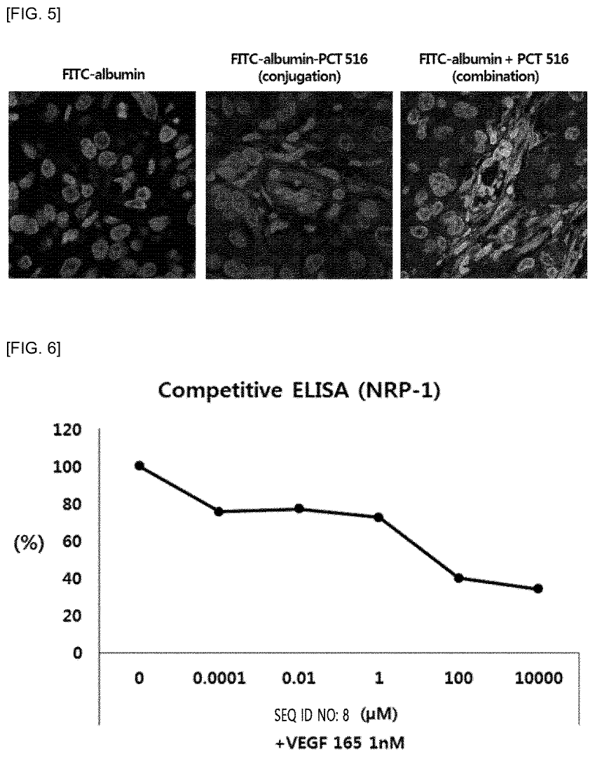

This result means that all drugs that are fused or mixed with the tumor tissue-penetrating peptide are delivered into the tumor, and that even drugs having a high molecular weight can be delivered (FIG. 5).

### Example 6: Effect of Tumor Tissue-Penetrating Peptide on Inhibiting Binding Ability of VEGF 165 to NRP-1

In order to examine the effect of the peptide of SEQ ID NO: 8 of the present invention on inhibiting the binding ability of VEGF 165 to NRP-1, NRP-1 was treated with the tumor tissue-penetrating peptide and VEGF and binding was examined by the competitive ELISA assay, and as a result, it has been found that NRP-1 binding of VEGF165 decreases when treatment with the tumor tissue-penetrating peptide is performed. Therefore, the tumor tissue-penetrating peptide is more likely to bind to NRP-1 competitively, and thus can interfere with neovascularization, and this means that the tumor tissue-penetrating peptide can suppress tumor growth (FIG. 6).

From the results described above, it has been found that the tumor tissue-penetrating peptide of the present invention, a derivative thereof, or a fragment thereof effectively delivers a drug deep into tumor or tissues through an assembling or fusion method and exhibits not only excellent penetration activity into biomembranes but also excellent tumor selectivity to maximize anticancer activity, and thus has a remarkable effect as an excellent tumor tissue-penetrating peptide.

From the description above, those skilled in the art to which the present invention pertains will be able to understand that the present invention can be implemented in other specific forms without changing its technical idea or essential features. In this regard, the embodiments described above should be understood in all respects as illustrative and not restrictive. The scope of the present invention should be construed as including the meaning and scope of the claims described below rather than the detailed description above, and all changes or modified forms derived from the equivalent concept thereof are included in the scope of the present invention.

1-26. (canceled)

27. A delivery method of a physiologically active substance to tumor, comprising administering a physiologically active substance and a tumor tissue-penetrating peptide consisting of any one of amino acid sequences of SEQ ID NO: 5 to SEQ ID NO: 8, a derivative thereof, or a fragment thereof to a subject.

28. The delivery method of a physiologically active substance to tumor according to claim 27, wherein the derivative has a substitution of an amino acid corresponding to any one position selected from the group consisting of 1st, 2nd, 5th, 6th, 9th, and 12th positions from an N-terminus of the tumor tissue-penetrating peptide consisting of any one of amino acid sequences of SEQ ID NO: 5 to SEQ ID NO: 8 with another basic amino acid.

29. The delivery method of a physiologically active substance to tumor according to claim 28, wherein the basic amino acid is lysine or arginine.

30. The delivery method of a physiologically active substance to tumor according to claim 27, wherein the derivative is any one or more selected from the group consisting of SEQ ID NO: 1 to SEQ ID NO: 4.

31. The delivery method of a physiologically active substance to tumor according to claim 27, wherein the tumor tissue-penetrating peptide consisting of any one of amino acid sequences of SEQ ID NO: 5 to SEQ ID NO: 8, the derivative thereof, or the fragment thereof allows a physiologically active substance to penetrate into a tumor tissue.

32. The delivery method of a physiologically active substance to tumor according to claim 27, wherein the tumor tissue-penetrating peptide is tumor selective.

33. The delivery method of a physiologically active substance to tumor according to claim 27, wherein the tumor tissue-penetrating peptide has binding ability to neuropilin 1 (NRP-1).

34. The delivery method of a physiologically active substance to tumor according to claim 27, wherein the physiologically active substance is directly or indirectly linked to the tumor tissue-penetrating peptide.

35. The delivery method of a physiologically active substance to tumor according to claim 27, wherein the tissue-penetrating peptide undergoes self-assembling or self-fusion with the physiologically active substance.

36. The delivery method of a physiologically active substance to tumor according to claim 27, wherein the tissue-penetrating peptide surrounds or fuse with the physiologically active substance.

37. The delivery method of a physiologically active substance to tumor according to claim 27, wherein the physiologically active substance is a drug, a natural product, a chemical compound, a protein, a peptide, an amino acid, an nucleic acid, a lipid, and a liposome.

38. The delivery method of a physiologically active substance to tumor according to claim 37, wherein the physiologically active substance is a drug.

39. The delivery method of a physiologically active substance to tumor according to claim 38, wherein the drug is for tumor suppression.

40. The delivery method of a physiologically active substance to tumor according to claim 27, which is for prevention or treatment of cancer or an angiogenesis-related disease.

41. The delivery method of a physiologically active substance to tumor according to claim 27, wherein the physiologically active substance is any one or more selected from the group consisting of doxorubicin, gemcitabine, taxol, and albumin.

42. A method for preventing or treating cancer or an angiogenesis-related disease, the method comprising administering a tumor tissue-penetrating peptide consisting of any one of amino acid sequences of SEQ ID NO: 5 to SEQ ID NO: 8, a derivative thereof, or a fragment thereof and a drug to a subject,
wherein the drug is for prevention or treatment of cancer or an angiogenesis-related disease.
